(19) United States
(12) Patent Application Publication
(10) Pub. No.: US 2012/0141431 A1
(43) Pub. Date: Jun. 7, 2012

(54) METHODS FOR IDENTIFYING, ISOLATING, AND UTILIZING ENDOCRINE PROGENITOR CELLS FROM ADULT HUMAN PANCREAS

(76) Inventors: Michael J. Shamblott, Baltimore, MD (US); Michael Cohen, West Orange, NJ (US)

(21) Appl. No.: 13/206,320

(22) Filed: Aug. 9, 2011

Related U.S. Application Data
(63) Continuation of application No. 11/980,797, filed on Oct. 31, 2007, now abandoned.
(60) Provisional application No. 60/855,374, filed on Oct. 31, 2006.

(51) Int. Cl. A61K 35/39; A61P 1/18; C12M 1/34; G01N 33/567; C12N 5/071

(52) U.S. Cl. 424/93.7; 435/7.21; 435/325; 435/287.2

(57) ABSTRACT

The presence of the cell surface marker CD133 or the presence of a glycosylated form of the prominin-1 gene product on adult pancreatic cells is used to identify pancreatic endocrine progenitor cells, and useful in methods of isolation and enrichment. Isolated pancreatic endocrine progenitor cells can be used for cell based therapy for insulin-dependent diabetes and pancreatectomy patients.

### CROSS-REFERENCE TO RELATED APPLICATION

This application claims priority under 35 U.S.C. §119(e) to U.S. provisional patent application Ser. No. 60/855,374, filed Oct. 31, 2006, which is incorporated herein by reference in its entirety.

### BACKGROUND

The human gene prominin 1 (PROM1) codes for a gene product having a glycosylated epitope designated CD133. The PROM1 gene product is a pentaspan transmembrane (5-TM) glycoprotein and belongs to a new molecular family of 5-TM proteins. This family includes members from several different species including human, mouse, rat, fly, and worm. The 5-TM structure includes an extracellular N-terminus, two short intracellular loops, two large extracellular loops and an intracellular C-terminus. PROM1 was initially shown to be expressed on primitive hematopoietic stem and progenitor cells and retinoblastoma and has since been shown to be expressed on hemangioblasts and neural stem cells as well as on apical surface protrusions of developing epithelium. The CD133 positive fraction of human bone marrow, cord blood and peripheral blood have been shown to efficiently engraft in xenotransplantation models, and have been shown to contain the majority of the granulocyte/macrophage precursors, NOD/SCID repopulating cells and CD34+ dendritic cell precursors. Phenotypically, CD133 positive cells in blood and marrow are CD34 bright, with CD34-dim CD71 bright cells being negative for CD133 expression. No natural ligand has yet been demonstrated for the CD133 molecule, and its function in hematopoietic tissue is unknown.

### SUMMARY OF THE INVENTION

In one embodiment, a method for identifying human pancreatic endocrine progenitor cells in a cellular sample or tissue sample is provided comprising exposing the cellular sample or tissue sample to at least one detectable agent that labels cells expressing a glycosylated form of a prominin 1 gene product, thereby identifying pancreatic endocrine progenitor cells therein. In another embodiment, a method for identifying human pancreatic endocrine progenitor cells in a cellular sample or tissue sample is provided comprising exposing the cellular sample or tissue sample to at least one detectable agent that labels cells expressing a glycosylated prominin-1 gene product epitope, termed CD133, thereby identifying pancreatic endocrine progenitor cells therein. In another embodiment, the at least one detectable label is a detectably labeled primary binding partner that binds to a glycosylated form of a prominin-1 gene product, or a combination of an unlabeled primary binding partner that binds to a glycosylated form of a prominin 1 gene product, and a detectably labeled secondary binding partner that binds to the unlabeled primary binding partner. The binding partners can be, by way of non-limiting examples, antibodies or lectins. In another embodiment, the at least one detectable agent is a detectably labeled primary antibody that binds to glycosylated prominin-1 gene epitope CD133/1 or CD133/2, or a combination of an unlabeled primary antibody that binds to epitope CD133/1 or CD133/2 and a detectably labeled secondary antibody that binds to the unlabeled primary antibody. In another embodiment, a primary antibody binds to at least one other glycosylated epitope of the prominin 1 gene product. In another embodiment, the detectable label is a fluorophore. In another embodiment, the detectably labeled primary antibody is a phycoerythrin-conjugated antibody to CD133/1 or a phycoerythrin-conjugated antibody to CD133/2. In another embodiment, at least one other marker is also detected. In another embodiment, the cellular sample is a pancreatic islet preparation. In another embodiment, the cellular sample is human pancreatic tissue.

In yet another embodiment, a method is provided for isolating or enriching human pancreatic endocrine progenitor cells from a cellular population comprising the steps of identifying cells expressing a glycosylated form of the prominin 1 gene product therein, then separating such cells from the cellular population, thereby isolating pancreatic endocrine progenitor cells or enriching the population thereof. In yet another embodiment, a method is provided for isolating or enriching human pancreatic endocrine progenitor cells from a cellular population comprising the steps of identifying CD133-expressing cells therein, then separating the CD133-expressing cells from the cellular population, thereby isolating pancreatic endocrine progenitor cells or enriching the population thereof. Non-limiting embodiments for identifying glycosylated prominin 1 gene product-expressing cells or CD133-expressing cells are described above. In one embodiment, the separating is performed by fluorescence activated cell sorting, utilizing, by way of non-limiting examples, antibodies including a detectably labeled antibody as described above. In another embodiment, the identifying and separating is by using a matrix to which CD133-binding moieties are bound, thereby binding cells expressing CD133 from a mixed population on the matrix. Such CD133-binding moieties include antibodies as mentioned above, lectins, or any other CD133 binding partner. In one such embodiment, magnetic beads bind to cells expressing CD133, magnetic beads to which CD133-expressing cells are bound are isolated, then the CD133-expressing cells bound thereto are released, thereby isolating CD133-expressing cells. In another embodiment, at least one additional marker is also used for the isolating or enriching. In one embodiment, the cellular population is obtained from a pancreatic islet preparation, which includes a mixture of islets and exocrine tissue. In another embodiment, the cellular population is obtained from human pancreatic tissue. In another embodiment, isolated or enriched human pancreatic endocrine progenitor cells are then expanded or cultured in vitro.

In still another embodiment, cells expressing a glycosylated form of a prominin 1 gene product that are isolated from pancreata as described herein are used for cell based therapies. In still another embodiment, CD133 expressing cells isolated from pancreata as described herein are used for cell based therapies. In another embodiment, CD133 expressing cells are used for ex vivo therapies. In another embodiment, CD133 expressing cells are used to prepare tissues for implantation. In another embodiment, the isolated cells are cultured or expanded in vitro before any of the aforementioned exemplary but non-limiting in vivo or ex vivo uses.

In a further embodiment, a method is provided for treating a patient in need of pancreatic endocrine cell replacement therapy such as a patient having insulin-dependent diabetes mellitus or following pancreatic resection, comprising isolating cells expressing a glycosylated form of a prominin 1 gene product from a cellular population, then administering the isolated pancreatic endocrine progenitor cells therein to the patient. In another embodiment, the cells are cultured or expanded in vitro before administration. In another embodiment the cellular population is from pancreatic tissue from one or more individuals. In a further embodiment, the cellular population is obtained from resected pancreatic tissue obtained from the same patient obtained during surgery.

In another embodiment, a system is provided for isolating or enriching pancreatic endocrine progenitor cells from a cellular sample, the system comprising means for detecting cells expressing a glycosylated form of a prominin-1 gene product therein, and means for separating the cells expressing a glycosylated form of a prominin-1 gene product therein from other cells in the cellular sample. In a further embodiment, means for detecting cells comprises detectable agent that labels cells expressing a glycosylated form of a prominin-1 gene product, such as described above. In another embodiment, means for separating such expressing cells is provided by, for example, fluorescence activated cell sorting or magnetic bead sorting, as described above.

In another embodiment, isolated pancreatic endocrine progenitor cells are provided that are prepared by the process of obtaining a cellular population comprising pancreatic cells, identifying cells expressing a glycosylated form or a prominin 1 gene product therein, then separating said cells from the cellular population; thereby providing isolated pancreatic endocrine progenitor cells. In another embodiment, the isolated pancreatic endocrine progenitor cells are subsequently cultured or expanded in vitro.

### BRIEF DESCRIPTION OF THE DRAWINGS

FIG. 1 depicts the change in the percentage and total number of CD133+ cells over time in three density fractions of two independent islet isolations. Days in culture indicated along X-axis. Mean percentage of CD133+ cells (black shade) and mean total number of CD133+ cells (unshaded) indicated along Y-axis as a percentage of mean baseline level on day 2. Significance determined by Student's t-test, ***, P<0.001, **, P<0.01. Relative contribution of each fraction to the change in total number of CD133+ cells indicated in inset. Fraction I (high islet purity, dark grey), II (medium grey) and III (low islet purity, light grey). Inset axes are identical to larger figure;

FIG. 2A-E show the effects of media composition and incubation temperature on CD133 expression. Y-axes, side scatter, media composition and days in culture, X-axes, CD133 intensity and culture temperature. CD133 indicated within gates of each panel. A, Day 3 in CMRL 1066 media (CMRL); B-C, CMRL media at 37° C. and 25° C., respectively; D-E, Miami media 1A (MM1A) at 37° C. and 25° C., respectively;

FIG. 3A-D depict the change in percentage of CD133+ cells over time in culture and with DAPT. A. Results from two independent islet preparations grown in MM1A and CMRL media combined and expressed on Y-axis as percentage of baseline which is the mean % CD133 on day 3, Days in culture (D3, D7) and concentration of DAPT indicated on X-axis, 0 is DMSO carrier alone. Significance determined by Student's t-test, **, P<0.01, **, P<0.05,*. B-C, Normalized mRNA level indicated along Y-Axis expressed as % of baseline, where baseline is the mean level on day 3. Levels in adult human pancreas (P), following culture (D3, D7) and concentration of DAPT indicated along X-axis. Significance determined by Student's t-test, ***, P<0.001, **, P<0.01. B, PTF1 (black shade) and PDX1 (grey shade); C, NGN3; D, normalized mRNA level in tissue cultured for 4 days (D4) and CD133-enriched population (D4 CD133+) indicated along Y-Axis, PTF1 (black shade), PDX1 (unshaded), NGN3 (gray shade);

FIG. 4 shows the expression of cytokeratin 19 (CK19) and amylase (AMY) protein over time and with N-[N-(3,5-difluorophenacetyl-L-alanyl)]-S-phenylglycine t-butyl ester (DAPT). % CK19+ (black shade) and AMY+ (gray shade) indicated on Y-axis. Results from two independent islet preparations grown in MM1A and CMRL media combined. Days in culture (D3, D7) and concentration of DAPT indicated on X-axis, 0 is DMSO carrier alone. Significance determined by Student's t-test, ***, P<0.001, *, P<0.05; and

FIG. 5 shows the percentage of CD133+ cells over time in culture and with DAPT and 7-amino-4-chloro-3-methoxyisocoumarin (JKL6). % CD133 in indicated on Y-axis. Days in culture (D3, D7) and concentration of DAPT and JKL6 indicated on X-axis, 0 is DMSO carrier alone.

### DETAILED DESCRIPTION OF THE EMBODIMENTS

The gene product of human prominin 1 (PROM1), a member of a molecular family of pentaspan transmembrane (5-TM) glycoproteins, is expressed widely on human adult and fetal tissues. CD133 is a glycosylated epitope of PROM1; antibodies against CD133 do not recognize PROM1 in most tissues or in differentiated cell types. However, antibodies to CD133 do recognize PROM1 in human fetal neural, kidney, muscle, skin, and hematopoietic stem cell, and CD133 is a marker for a progenitor cell population. In searching for progenitor cells potentially capable of differentiating into pancreatic endocrine tissues and in particular into insulin-producing cells for the treatment of insulin-dependent (type 1, or juvenile) diabetes mellitus and other pancreatic diseases including pancreatectomy-induced diabetes, the inventors herein discovered CD133-expressing cells in adult human pancreata and in enriched pancreatic islet preparations, which comprise islets and exocrine tissue.

Islet preparations are often maintained in culture for several hours to days prior to allotransplantation. In one embodiment herein, CD133+ cells were abundant in every preparation, ranging from ˜3% to over 30% after 48 hrs or less of culture and exceeded 50% following 4 days of culture.

Thus, in one embodiment, the expression of CD133 on a mixed population of pancreatic cells can serve as a means for identifying, quantitating, localizing, isolating or enriching such cells from a population, the latter providing ample material for cell-based therapy for insulin-dependent diabetes or other pancreatic diseases. In another embodiment, the expression of a glycosylated form of a prominin 1 gene product on a mixed population of pancreatic cells can serve as a means for identifying, quantitating, localizing, isolating or enriching such cells from a population, the latter providing ample material for cell-based therapy for insulin-dependent diabetes or other diseases of the pancreas. In another embodiment, the mixed pancreatic cell population comprises islets and exocrine tissue. In another embodiment, the cells are found in pancreatic exocrine tissue.

As mentioned above, CD 133 comprises epitopes of a glycosylated form of a prominin-1 gene product, and the methods described herein are inclusive of all forms and variants of such epitopes and glycosylated forms of prominin-1 gene products, such as allelic variations, splice variants, the corresponding gene or protein from another species, differentially glycosylated forms, mutated or altered forms, for example, by substitution of variant amino acid residues from one species in the polypeptide of another, so as to correspond to an analogous gene or protein as if from another species, as a progenitor cell marker. Non-limiting examples of splice variants are described in Fargeas et al., J Cell Sci. 2004 117:4301-11. Alternate glycosylation patterns of the prominin 1 gene product characteristic of progenitor cells are also embodied herein, wherein a variant of the eight N-glycosylation sites is provided, such as that discussed by Fargeas et al., Future Lipidology 2006, 1:213-225. Variant is meant to include modifications that retain the same essential character of the parent protein. In one embodiment, proteins with greater than about 80%, preferably at least 90% and particularly preferably at least 95% homology with the prominin 1 gene product are considered as variants. Variants may include the deletion, modification or addition of single amino acids or groups of amino acids within the protein sequence.

Thus, in one embodiment, the detectability of CD133 expressed on cells in a population of pancreatic cells serves as a marker for the presence and/or location of pancreatic endocrine progenitor cells in the population. In another embodiment, the presence of CD133 on pancreatic endocrine progenitor cells provides a means for isolating or enriching such cells for performing studies thereon including providing cell-based therapies. In one embodiment, the cell-based therapy is a treatment for insulin-dependent diabetes. In another embodiment the cell-based therapy is a treatment for patients who have become insulin dependent as a result of having the pancreas resected, for example, to treat pancreatitis. The detection methods can be qualitative or quantitative, and provide specific locations of such cells or the relative or absolute number of such CD133-expressing cells in a particular population of cells or in tissues.

Methods for identifying CD133 expressing cells can be based on any number of methods known in the art. Among the various methods for detecting cells expressing a specific marker, some methods are typically used if the cells are to remain viable following detection, such as for further in vitro study or for transplantation or implantation into a patient, and other methods render the identified cells less amenable to further uses in their living state, for example, in studying pathology specimens or for studies at the termination of cell based or in vivo studies. The methods herein are not so limiting and applications maintaining the viability of living cells as well as those preserving cells are fully embodied herein.

Methods of identifying CD133 expressing cells can be based on, by way of non-limiting examples, localizing or quantitating CD133 epitopes on the surface of the cells, or localizing or quantitating CD133 epitopes within the cytoplasm or subcellular compartments therein. Exemplary methods for the aforementioned localizing or detecting are provided below but are not intended to be limiting in any way.

Detection methods are in one embodiment based upon the detection of the binding of a binding partner to a cell expressing a glycosylated form of a prominin 1 gene product. Binding partners can be detectably labeled, or can be unlabeled but further detectable by another binding partner that is detectably labeled and binds thereto. Such uses of binding partners such as antibodies, including labeled primary antibodies and labeled lectins are known in the art. Moreover, combination systems of unlabeled primary antibodies and labeled secondary antibodies are also well known in the art. Such dual systems can also include two antibodies, lectins, avidin-biotin systems, antibodies to labels, and include amplification systems to increase the detection signal. As will be described below, such detection systems are useful not only for identifying the expression of a gene product but also in isolating cells expressing such a gene product utilizing selective binding to a matrix such as a resin or beads. The invention is not so limiting as to the means for detecting the expression of the glycosylated form of a prominin 1 gene product and is inclusive of all such means.

Antibody-based detection methods are among those typically but not always used to identify expression of a protein or an epitope thereof by cells, regardless of whether cells require viability during or after detection. The antibody can be a monoclonal or polyclonal antibody. Ready guidance from the literature can be followed to prepare such antibodies that specifically bind to CD133 or other epitopes of the prominin 1 gene product on the cell surface, and can be used on living cells to detect CD133 on the cell surface, or in sectioned cells or tissue specimens to detect CD133 on the surface.

In one embodiment, the term “antibody” includes complete antibodies (e.g., bivalent IgG, pentavalent IgM) or fragments of antibodies in other embodiments, which contain an antigen binding site. Such fragment include in one embodiment Fab, F(ab′)₂, Fv and single chain Fv (scFv) fragments. In one embodiment, such fragments may or may not include antibody constant domains. In another embodiment, F(ab)'s lack constant domains which are required for complement fixation. scFvs are composed of an antibody variable light chain (V_{L}) linked to a variable heavy chain (V_{H}) by a flexible linker. scFvs are able to bind antigen and can be rapidly produced in bacteria. The invention includes antibodies and antibody fragments which are produced in bacteria and in mammalian cell culture. An antibody obtained from a bacteriophage library can be a complete antibody or an antibody fragment. In one embodiment, the domains present in such a library are heavy chain variable domains (V_{H}) and light chain variable domains (V_{L}) which together comprise Fv or scFv, with the addition, in another embodiment, of a heavy chain constant domain (C_{H1}) and a light chain constant domain (C_{L}). The four domains (i.e., V_{H}-C_{H1} and V_{L}-C_{L}) comprise an Fab. Complete antibodies are obtained in one embodiment, from such a library by replacing missing constant domains once a desired V_{H}-V_{L} combination has been identified.

The antibodies described herein can be monoclonal antibodies (Mab) in one embodiment, or polyclonal antibodies in another embodiment. Antibodies which are useful for the methods described herein can be from any source, and in addition may be chimeric. In one embodiment, sources of antibodies can be from a chicken, mouse, rat, sheep, goat, horse, or a human in other embodiments. Secondary antibodies are typically antibodies that bind to another antibody, and are typically prepared in a species different from the originating species of the primary antibody, such that, for example, the secondary antibody may be a rat anti-mouse antibody, or a goat anti-rat antibody, or vice versa, e.g., mouse anti-rat antibody. In some cases a secondary antibody may be directed against a moiety conjugated to the primary antibody, such as a fluorescent moiety. In other embodiments, other binding partners such as avidin and biotin may be employed. In certain embodiments, a detectable primary antibody is used in the detection. In other embodiments, and in particular where amplification of the detectable signal that indicates the presence of CD133 is needed, secondary antibodies or even further amplification techniques can be used to increase the detectability of the extent of binding of the primary antibody can be employed. Such amplification systems are well known in the art.

The detection agent described herein can be a lectin or combination of lectins selected or designed to specifically bind to the CD133 glycan structure. These lectins can be in solution, detectably labeled or detected or retrieved by a secondary detection antibody or preferably, be attached to a solid substrate such as a magnetic bead or other surfaces that can be used to retrieve cells.

Detection of antibody binding to a cell typically requires a detectable label, either directly bound to the CD133-binding antibody (primary antibody) itself, or the detectable label can be present on a secondary antibody that binds to the primary antibody. Various detectable labels are embodied herein, and the selections are not intended to be limiting. Labels such as fluorescent moieties, radioactive elements and compounds, and proteins or other entities with enzymatic activity have been used in the art and are well known, and are applicable to different methods of detection. In one embodiment, among useful fluorescent labels is phycoerythrin. In another embodiment, radioactive labels include ¹²⁵I.

As noted above, the term “detectable label” or “detectably labeled” refers in one embodiment to a composition or moiety that is detectable by spectroscopic, photochemical, biochemical, immunochemical, electromagnetic, radiochemical, or chemical means such as fluorescence, chemifluoresence, or chemiluminescence, or any other appropriate means. In another embodiment, detectable labels are fluorescent dye molecules, or fluorophores, such fluorescein, phycoerythrin, CY3, CY5, allophycocyanine, Texas Red, peridenin chlorophyll, cyanine, FAM, JOE, TAMRA, TET, and VIC, by way of non-limiting examples.

For example, Miltenyi Biotec (Auburn, Calif.) sells antibody-based reagents for identification and isolation of CD133 expressing cells; antibodies include clone AC133 (mouse IgG1), 293C3 (mouse IgG2b), and AC141 (mouse IgG1). These antibodies recognize two different epitopes CD133/1 (clone AC133) and CD133/2 (clone 293C3 and clone AC141), respectively, on the CD133 molecule. In alternate embodiments, antibodies can be raised to other glycosylated epitopes of the prominin 1 gene product.

Thus, in one embodiment, a labeled primary antibody that binds to CD133/1 or CD133/2, or a combination of an unlabeled primary antibody that binds to CD133/1 or CD133/2 and a labeled secondary antibody that binds to the unlabeled primary antibody, can be used to identify CD133 expressing cells. In another embodiment, a phycoerythrin-conjugated antibody to CD133/1 or a phycoerythrin-conjugated antibody to CD133/2 is used. Using a fluorescent label such as phycoerythrin (PE), CD133 expressing cells can be identified using fluorescence microscopy. In other embodiments, a biotinylated primary antibody and a detectable reagent that binds to biotin, such as a fluorescent- or enzyme-conjugated streptavidin or other avidin derivative, can be used for fluorescence localization, immunohistochemical localization or detection by light microscopy. As will be seen below, an advantage of using phycoerythrin is that it is both detectable (fluorescent), and an antibody can be raised thereto, the anti-phycoerythrin antibody useful as an affinity reagent to isolate cells to which phycoerythrin is bound, via for example using the aforementioned phycoerythrin-conjugated anti-CD133 antibody. The anti-phycoerythrin antibody can be of the same species or of a different species as the primary anti-CD133 antibody.

In yet another embodiment, localization of CD133 expressing cells in a cellular or tissue sample can be performed using immunohistochemical techniques whereby, for example, whole cells or thin sections of tissue are stained with reagents that identify CD133 epitopes, such as antibodies as described above either directly labeled or by using a labeled secondary antibody that produces a visible product, for example, through an enzymatic reaction, at the sites of CD133. Such immunohistochemical localization methods are well known in the art and can be readily applied to CD133.

Sources of pancreatic cells for the methods described herein include pancreatic islet preparation, i.e., cells isolated from the islets of human or other species pancreata, or cells prepared from human pancreatic tissue. Tissues from adults as well as those from fetal sources are embraced herein. Pancreatic islet cell preparations, which comprise islets and exocrine tissue, can be obtained from any of a number of academic and/or clinical islet purification services. For patients undergoing pancreatectomy for the purpose, for example, of treatment of pancreatitis, the patient's own resected pancreas tissue can provide the source of cells from which pancreatic endocrine progenitor cells can be isolated by the methods embodied herein then administered to the same patient, or to another patient for the treatment of, for example, diabetes mellitus. And likewise, a pancreatectomy patient can be administered autologous pancreatic endocrine progenitor cells from a single unrelated individual or a pool of individuals.

In one embodiment, pancreatic endocrine progenitor cells herein are cells from the adult human pancreas that express CD133. In another embodiment, the pancreatic endocrine progenitor cells herein are cells from the adult human pancreas that express a glycosylated form of the prominin-1 gene product. In another embodiment, the pancreatic endocrine progenitor cells herein are cells found in the exocrine tissue of adult human pancreata that express a glycosylated form of the prominin-1 gene product. In another embodiment, the pancreatic endocrine progenitor cells herein are CD133 expressing cells are found in the exocrine tissue of adult human pancreata.

The aforementioned exemplary methods for identifying cells expressing CD133, and in particular methods that do not impact the viability of the cells, readily lend themselves to methods for isolating from a mixed cellular population cells that express CD133. Thus, in another embodiment, CD133 expressing cells are isolated from or enriched within a mixed cellular population, utilizing various methods of detecting the expression of CD133 on the cell surface. By way of non-limiting examples, fluorescence activated cell sorting technology can be used. The various reagents mentioned above useful for identifying cells expressing CD133 in pancreatic tissue are also useful as reagents for separating such cells from a mixed cellular population, such as by binding to a solid matrix or using magnetic bead technology. Anti-CD133 antibodies are but one example of the use of a CD133 binding partner for isolating or separating CD133 expressing cells.

Thus, in one embodiment, fluorescence activated cell sorting (FACS) techniques can be used to isolate cells expressing CD133, using either a primary anti-CD133 antibody conjugated to a fluorescent moiety, or an unlabeled or nonfluorescently-labeled primary anti-CD133 antibody and a secondary antibody conjugated to a fluorescent moiety or a fluorescent reagent that binds to the primary antibody or by using a lectin that recognizes CD133 glycans. Other binding pairs such as biotin and avidin can be used to achieve the same desired cell labeling. FACS methodology is well known in the art.

In another embodiment, cells expressing CD133 can be directly isolated from a mixed population using a matrix or surface to which an antibody to CD133 is conjugated, such that CD133 expressing cells bind to the matrix or surface, non-adherent cells can be washed away, and the CD133 expressing cells eluted from the matrix or surface. In one embodiment, a matrix such as agarose or Sepharose in the form or beads can be conjugated with antibodies to CD133. CD133 expressing cells in a mixed population are exposed to the matrix, by admixing therewith or passage through a column thereof, to which CD133 expressing cells adhere, then the matrix can be washed and the cells eluted therefrom using a high salt or low pH elution buffer, or other methods that interfere with antibody-epitope interaction or methods that act to cleave the connection between the bead and desired cell type. Such methods and reagents therefor are well known in the art. In another embodiment, magnetic beads to which anti-CD133 antibodies are conjugated are used to bind CD133 expressing cells, after which the beads are separated based on their magnetic properties, washed and the CD133 expressing cells eluted therefrom. Such magnetic beads are available from Miltenyi Biotec, and methods of use described in the manufacturer's instructions. In yet another embodiment, agarose or Sepharose beads to which lectins are attached are used to bind CD133 expressing cells. CD133 expressing cells in a mixed population are exposed to the matrix, by admixing therewith or passage through a column thereof, to which CD133 expressing cells adhere, then the matrix can be washed and the cells eluted therefrom using a unconjugated glycan to that interferes with the CD133-lectin interaction or methods that act to cleave the connection between the bead and desired cell type. Such methods and reagents therefor are well known in the art

In other embodiments, matrix or magnetic bead separation can be achieved using a secondary antibody conjugated to the matrix or beads, the secondary antibody directed against a primary antibody that binds to CD133. For example, in one embodiment, after use of a primary antibody that binds to CD133/1 or CD133/2 that is labeled with phycoerythrin, magnetic beads or a matrix conjugated with an antibody that binds to phycoerythrin can used to bind CD133 expressing cells, after which the beads can be washed and the CD133 expressing cells released. For example, Miltenyi Biotec sells magnetic beads conjugated to an anti-phycoerythrin antibody (Anti-PE microbeads). Alternately, a secondary antibody against the primary antibody molecule can be used. There methods are merely illustrative of affinity procedures and variations thereof are well known in the art and are fully embraced herein.

In embodiments of the methods for isolation of or enrichment for CD133 expressing cells from a cellular population, the cellular population can be obtained from a pancreatic islet preparation, or from human pancreatic tissue. As noted above, pancreatic islet cell preparations can be obtained from any of a number of academic and/or clinical islet purification services. Adult as well as fetal tissues are embraced herein.

In any of the embodiments described herein, the isolated or enriched CD133 expressing cells can be cultured or expanded in vitro prior to any of the various uses described herein, among others, in order to, by way of non-limiting example, expand or increase the population of cells or increase the expression of CD133 thereon. For example, cells enriched or isolated in accordance with the teaching herein may be cultured in Miami Medium 1A, or in HuES medium [KO-DMEM, 1× penicillin/streptomycin, 1X glutamax, 1× NEAA, 10% KO serum replacement, 0.1× 2-mercaptoethanol, 1× N2 supplement (Invitrogen), 10% Plasmanate (Bayer), fibroblast growth factor 2 (20 ng/ml), leukemia inhibitory factor, (10 ng/ml), epidermal growth factor (20 ng/ml)], or in PS medium [DMEM/F12, 1× penicillin/streptomycin, 1× glutamax, 1× N2 supplement (Invitrogen), fibroblast growth factor 2 (20 ng/ml), leukemia inhibitory factor, (10 ng/ml), epidermal growth factor (20 ng/ml)] that has been conditioned by overnight incubation with human embryoid body derived cell line SDEC (Shamblott et al., Proc Natl Acad Sci USA. 2001; 98:113-8). In another embodiment, expansion of these cells is achieved by the inclusion of platelet derived growth factor-BB (PDGF-BB) in the culture medium. Thus, in one embodiment, an expanded population of pancreatic endocrine progenitor cells is provided by isolating or enriching CD133 expressing cells as described herein, then expanding the enriched or isolated cells in culture. The foregoing examples of culture media and conditions are merely exemplary and non-limiting.

In yet another embodiment of the invention, methods are provided for treating a patient having insulin-dependent diabetes mellitus using CD133 expressing cells isolated from a cellular population in accordance with, and by way of non-limiting examples, the embodiments described above, then administering the isolated pancreatic endocrine progenitor cells to the patient. In one embodiment the cells are cultured or expanded in vitro prior to use, such as by one of the methods described above. Patients with type 1 diabetes mellitus, also known as juvenile diabetes or insulin-dependent diabetes mellitus (IDDM) no longer produce adequate levels of insulin as a consequence of the depletion of insulin-producing cells in the pancreatic islets through auto-immune or other causes. Restoration of glycemic control in such patients by exogenous insulin administration, timed to meals and other fluctuations in blood glucose levels, prevents acute hyperglycemic crises and staves off development of macrovascular and microvascular complications, but a pancreas transplant restores normoglycemia without the requirement for insulin injections and frequent blood glucose level monitoring. Unfortunately, there are not adequate pancreata available for transplant. The embodiments herein provide ample material for cell-based therapy for insulin-dependent diabetes.

For example, CD133 expressing cells isolated or enriched in accordance with the embodiments herein can be directly injected into the hepatic duct or the associated vasculature of a patient. In another embodiment the cells can be cultured and expanded in vitro prior to injection. Similarly, cells can be delivered into the pancreas by direct implantation or by injection into the vasculature. Cells engraft into the liver or pancreatic parenchyma, taking on the functions normally associated with pancreatic cells, respectively. Moreover, before implantation or transplantation the cell obtained as described herein can be genetically manipulated to reduce or remove cell-surface molecules responsible for transplantation rejection in order to generate universal donor cells. For example, the mouse Class I histocompatibility (MHC) genes can be disabled by targeted deletion or disruption of the beta-microglobulin gene (see, e.g., Zijlstra, Nature 342:435-438, 1989). This allows indefinite survival of murine pancreatic islet allografts (see, e.g., Markmann, Transplantation 54:1085-1089, 1992). Deletion of the Class II MHC genes (see, e.g., Cosgrove, Cell 66:1051-1066, 1991) further improves the outcome of transplantation. The molecules TAP1 and Ii direct the intercellular trafficking of MHC class I and class II molecules, respectively (see, e.g., Toume, Proc. Natl. Acad. Sci. USA 93:1464-1469, 1996); removal of these two transporter molecules, or other MHC intracellular trafficking systems may also provide a means to reduce or eliminate transplantation rejection. Such techniques can be applied to human cells and the corresponding HLA antigens. In another embodiment, the cellular population is obtained from a pancreas HLA matched to the subject.

In yet another embodiment of the invention, methods are provided for treating a patient having chronic pancreatitis using CD133 expressing cells isolated from a cellular population in accordance with and by way of non-limiting examples, the embodiments described above, then administering the isolated pancreatic endocrine progenitor cells to the patient. In one embodiment the cells are cultured or expanded in vitro prior to use. Patients with chronic pancreatitis require removal of their pancreas to alleviate pain. Pancreatic resection results in insulin-dependent diabetes unless islets are purified from the resected tissue and transplanted back to the patient. As a result of pancreatitis, resected pancreata frequently do not contain sufficient islets to restore patients to normoglycemia following transplantation, resulting in restoration of glycemic control in such patients by exogenous insulin administration, timed to meals and other fluctuations in blood glucose levels, prevents acute hyperglycemic crises and staves off development of macrovascular and microvascular complications. For example, CD133 expressing cells isolated or enriched in accordance with the embodiments herein can be directly injected into the hepatic duct or the associated vasculature of a patient. In another embodiment the cells can be cultured and expanded in vitro prior to injection. Cells engraft into the liver parenchyma, taking on the functions normally associated with pancreatic cells, respectively. In the case of chronic pancreatitis, the CD133 expressing cells could be autologous, and would therefore require no additional modification to, or match of the HLAs prior to transplantation. In other embodiments, the transplant is allogeneic.

In another embodiment, a system is provided for isolating or enriching pancreatic endocrine progenitor cells from a cellular sample comprising:
a. means for detecting cells expressing a glycosylated form of a prominin-1 gene product therein, and
b. means for separating the cells expressing a glycosylated form of a prominin-1 gene product therein from other cells in the cellular sample.

Means for detecting cells expressing a glycosylated form of a prominin-1 gene product can be carried out by any of the methods described above, such as by use of a detectable agent that labels cells expressing a glycosylated form of a prominin-1 gene product. Such agents can include a labeled primary antibody that binds to a glycosylated form of a prominin-1 gene product, or a combination of an unlabeled primary antibody that binds to a glycosylated form of a prominin-1 gene product and a labeled secondary antibody that binds to the unlabeled primary antibody. Typically, the label is a fluorophore. One detectable agent can be a labeled primary antibody that binds to epitope CD133/1 or CD133/2, or a combination of an unlabeled primary antibody that binds to CD133/1 or CD133/2 and a labeled secondary antibody that binds to the unlabeled primary antibody.

Means for separating can be performed by fluorescence activated cell sorting, or by using magnetic beads that bind to cells expressing CD133, isolating the magnetic beads to which CD133-expressing cells are bound, then releasing the CD133-expressing cells bound thereto, thereby isolating CD133-expressing cells. Exemplary but non-limiting methods and reagents for carrying them out are described hereinabove.

In another embodiment, isolated pancreatic endocrine progenitor cells are described that are prepared by the process of obtaining a cellular population comprising pancreatic cells, detecting CD133-expressing cells therein, then separating said detected CD133-expressing cells from the cellular population; thereby providing isolated pancreatic endocrine progenitor cells. Exemplary embodiments of the process are described hereinabove. In one embodiment, detecting comprises exposing the cellular population to at least one detectable agent that labels cells that express CD133. In another embodiment, the at least one detectable agent is a labeled primary antibody that binds to CD133/1 or CD133/2, or a combination of an unlabeled primary antibody that binds to CD133/1 or CD133/2 and a labeled secondary antibody that binds to the unlabeled primary antibody. In yet other embodiments, the separating is performed by fluorescence activated cell sorting or using magnetic beads that bind to cells expressing CD133. In another embodiment, the labeled primary antibody is phycoerythrin-labeled anti-CD133/1 antibody or a phycoerythrin-labeled anti-CD133/2 antibody, and the magnetic beads are coated with an anti-phycoerythrin antibody. In another embodiment, the cellular population is obtained from a pancreatic islet preparation, or from human pancreatic tissue. In another embodiment, CD133 expressing cells are separated based on lectin binding. In any of the embodiments, the isolated pancreatic endocrine progenitor cells can be cultured in vitro to expand the population.

Methods for preparing an islet preparation from human pancreatic tissue are known in the art, for example, Ricordi, C., Pancreatic Islet Cell Transplantation. Austin R. G. Landes Co. 1992; 99-112. Such techniques can be applied to human donor material to provide cells for transplantation. CD133 positive cells have been identified in all 3 fractions typically isolated. The percentage of CD133+ cells increases in approximately one week of culture in Miami Media 1A, and tapers off by week two in culture. A culture period and media typically is described in standard operating procedures of islet transplant programs. Thus, CD133 immunoreactive cells can be efficiently isolated from clinical grade islet preparations.

### EXAMPLES

The following examples are intended to illustrate but not limit the invention. While they are typical of those that might be used, other procedures known to those skilled in the art may alternatively be used.

### Example 1

### Research Design and Methods

Islet culture. Islet preparation tissues resuspended in Miami Media 1 (Mediatech, Herndon, Va.) supplemented with 0.01 g/L glutathione or CMRL-1066 (Invitrogen) media supplemented with 10% fetal bovine serum were plated in low adhesion dishes at 37° C., or 25° C. Media was replaced every 2-3 days. Islet purity was assessed by staining in 0.25 mg/ml dithizone. Inhibition of gamma-secretase was by daily addition of DAPT (N-[N-(3,5-difluorophenacetyl-L-alanyl)]-S-phenylglycine t-butyl ester) or JKL6 (7-amino-4-chloro-3-methoxyisocoumarin) prepared in DMSO. Bromodeoxyuridine (BrdU) was added daily to 10 micromolar.

Reverse transcriptase polymerase chain reaction (RTPCR). Synthesis of cDNA was performed by using oligo (dT) primers in a standard reverse transcriptase reaction. Levels of HES 1, PTF1, PDX1, NGN3 and CYCLOPHILLIN A were determined by using Taqman® gene expression assays Hs00172878_m1, Hs00603586_g1, Hs00426216_m1, Hs00360700_g1 and Hs99999904_m1, respectively. Mean levels (3-6 readings/sample) of genes of interest were normalized to mean levels of CYCLOPHILLIN A. Significance was determined by 2-tailed heteroscedastic Student's t-test. Human adult and fetal pancreas RNA was commercially prepared.

Immunochemistry. Five micron frozen sections of normal human adult pancreas (2 individuals) and islet preparation tissues were fixed for 5 min. in 1-4% paraformaldehyde prepared in Dulbecco's phosphate buffered saline (DPBS) and quenched for 5 min. in 50 mM glycine in DPBS then blocked in 5-10% serum, 1% BSA in DPBS for 30 min. at room temperature. 0.2% Triton-X100 was included for cytoplasmic and nuclear antigens except NGN3/CD133, PDX1/CD133 and NGN3/CA19.9 costaining, which was performed in its absence. Primary antibodies were diluted in blocking buffer; mouse anti-CD133/1 and CD133/2 unconjugated and conjugated to phycoerythrin (Miltenyi Biotec, Aubern, Calif., 1:10), mouse anti-human CD31 conjugated to fluorescein isothiocyanate (BD Biosciences, San Jose, Calif., 1:10), mouse anti-CK19 (Chemicon, Temecula, Calif., 1:300), mouse anti-CA19.9 (US Biological, Swampscott, Mass., 1:300), guinea pig anti-swine insulin (Dako, Carpinteria, Calif., 1:500), rabbit anti-PDX1 (Chemicon 1:250), goat anti-AMY (Santa Cruz, 1:300), mouse anti-mouse NGN3 (Developmental Studies Hybridoma Bank, Iowa City, Iowa, unconcentrated hybridoma conditioned media, 1:10). BrdU detection was by kit (BD Bioscience). Quantitative analyses of AMY and CK19 expression were carried out by imaging >600 nuclei for each treatment group. Quantitative analysis of NGN3 expression was carried out by imaging >800 nuclei for each treatment group.

Electron Microscopy. Formaldehyde fixed tissue in 10% gelatin was cryoprotected in 2.3M sucrose in 20% polyvinylpyrrolidone overnight at 4° C. Ultrathin sections were blocked in 10% FBS in DPBS for 30 min then incubated in anti-CA19.9 and anti-CD133/1 conjugated to magnetic beads (−50 nm diameter) diluted as above. CA19.9 was detected with 12 nm diameter colloidal gold goat anti-mouse IgM mu chain (Jackson ImmunoResearch, West Grove, Pa., 1:20) for one hour. CD133/1 was detected directly. Contrasting was done by incubation in 2% methyl cellulose and 0.3% uranyl acetate for 10 minutes at 4° C.

FACS, Cytospin and Magnetic bead isolation. Tissue rinsed in calcium/magnesium free DPBS was dissociated by incubation in 0.05% trypsin/EDTA for 5 min. at 37° C. Following trypsin neutralization and DNA removal by incubation with 120 units DNase I per ml prepared in 0.1% BSA, 1 mM CaCl₂, 0.5 mM MgCl₂, 0.6% Na citrate at room temperature for 2 min., single cells were collected by centrifugation at 200×g for 5 min. Cells were resuspended in degassed 0.5% BSA, 2 mM EDTA prepared in calcium/magnesium free DPBS and counted by Nucleocounter (New Brunswick Scientific, Edison, N.J.). For FACS analyses, primary antibodies were CD133/1 and CD133/2 (IgG2b, Miltenyi Biotec) conjugated to phycoerythrin. Isotype negative control antibodies were IgG1-PE and IgG2b-PE (BD Bioscience). CD133 expressing cells were purified by using the CD133/1 conjugated to magnetic beads (Miltenyi Biotec). Purification efficiency was determined by staining with CD133/2 conjugated to phycoerythrin. Dissaggregated cells were affixed to glass slides by using a Cytospin rotating at 8500 rpm for 4 min.

Time course culture and counting. 1.5 ml of tissue was removed from a 10 ml culture for FACS and histology for each time point. Volume was not replaced but media was added to adjust for evaporation. Cell count variability was determined by triplicate independent withdrawal and dissociation from a parallel islet preparation. The number of CD133 cells was calculated as total cell number x % CD133+.

Culture of CD133-enriched cells. Tissue was disaggregated and CD133+ cells were isolated by using immunomagnetic beads to >90% purity. 1.5×10⁴ CD133+ cells were plated by centrifugation at 200×g for 5 min. into wells of a 48-well plate coated with 50 mcg/ml type I bovine collagen and grown in DMEM/F12 media supplemented with 1% fetal calf serum, 100 U/ml penicillin, 100 mcg/ml streptomycin, 40 ng/ml human recombinant leukemia inhibitory factor, 50 ng/ml human recombinant epidermal growth factor and 50 mcg/ml Geneticin™ sulfate. Media was replaced every 3 days. Plating efficiency was calculated from 4 random fields of >400 cells total. Cell proliferation was calculated by counting a total of ˜300 4′,6-diamidino-2-phenylindole (DAPI) stained nuclei in isolated colonies from 10 random fields.

### Example 2

### CD133 is Expressed in the Adult Pancreas and Purified Islet Preparations

In the adult pancreas CD133 is expressed primarily by CK19 expressing ductal epithelial cells. Cell coexpressing CA 19.9 can be identified, but both antigens have distinct expression patterns. No co-expression of CD133 or CD31 (vascular endothelia marker) and insulin was detected and very little intra-islet expression of CD133 staining was observed. Other areas of punctuate immunoreactivity were apparent in most sections, possibly due to expression by acinar cells or pancreatic mesenchyme.

Human islet preparations containing a mixture of islets and co-purifying exocrine and duct tissue were classified by time in culture following initial organ digestion (specified here as day 0, D0) and also by purity expressed as percent islets. Preparations were cultured under standard conditions used to maintain islets for allotransplantation. By D4, islet preparations contain CD133+, CA19.9+ and CD133+/CA19.9+ cells. Electron microscopic analysis of this islet preparation (50% islets) on D7 indicated sporadic low level staining throughout the tissue and strong staining of epithelial cells lining the ductal lumen. Within the lumen, CD133 is expressed exclusively on microvilli, while CA19.9 is expressed on microvilli and planar regions of the apical domain. CD133 and CA19.9 have overlapping subcellular localization on cells which express both glycoproteins. On D7, ˜45% of cells were CD133+ (404/901 nuclei), ˜57% of cells were CA19.9+ (510/901 nuclei) and ˜63% of CA19.9+ cells were CD133+ (321/510 nuclei).

### Example 3

### The Percentage and Total Number of CD133+ Cells Increases in Culture

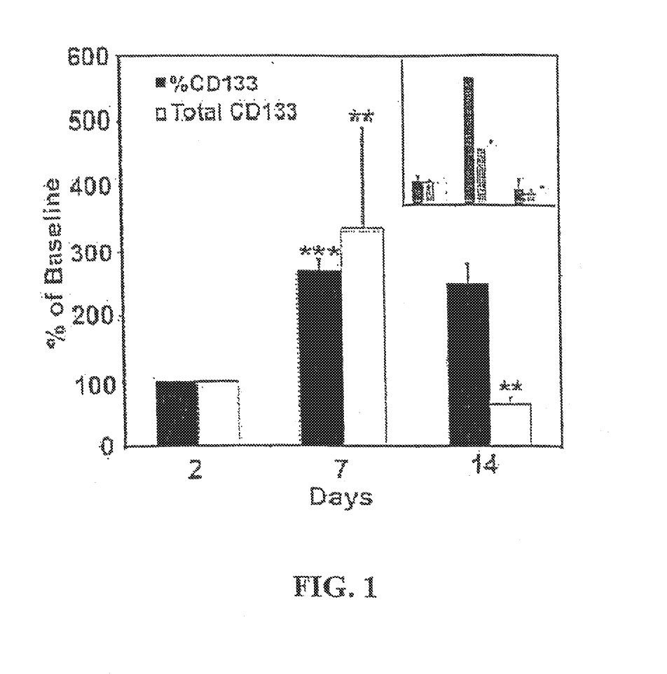

The expression of CD133 was examined on D2, 7 and 14 in three Ficoll density fractions of two independent islet isolations. On D2 of the first islet preparation, the percentage of cells expressing CD133 was 2.9, 13.2 and 12.6 from Ficoll fraction I (>95% islets), II (65% islets) and III (25% islets) respectively. The second islet preparation fractions had 90%, 60% and 30% islets, respectively. By D7, the mean percent (2.7-fold, P<0.001) and mean total number (3.3-fold, P<0.01) of CD133+ cells increased significantly compared to D2 (FIG. 1). Although the total number of CD133+ cells increased in all three fractions, the percent increase was highest for fraction I (˜5-fold) due to low levels at D2 (FIG. 1 inset). The total number of CD133+ cells in fraction I was consistently lower than the other fractions. Between D7 and 14 the total number of CD133 decreased significantly (5.1-fold, P<0.01). The decrease in % CD133 cells was not significant over this period.

### Example 4

### The Increased Percentage of CD133+ Cells is Not Due to CD133+ Cell Proliferation

Increased CD133 expression from D2 to 7 by immunohistochemistry is in agreement with the FACs results and also revealed a difference in subcellular localization of CD133. On D2, the expression of CD133 is primarily restricted to luminal surfaces while on D7, there is a broadened pattern that includes other membrane surfaces and/or cytoplasmic expression. Very little BrdU incorporation was detected on D7, with 1% (9/893), 1.7% (14/800) and 2.8% (30/1049) of nuclei staining positive in sections of FRI, II and III, respectively. No CD133+ cells were BrdU+ labeled. In contrast, virtually 100% of cells were BrdU labeled in an identically dosed control culture of rapidly proliferating human cells, insuring that any proliferation of CD133+ cells was not missed due to a short half-life of BrdU (data not shown). In contrast to scant intra-islet expression of CD133 in intact pancreas, on D7 many examples of CD133 expression were observed within areas of insulin expression in a culture that began as >95% islets.

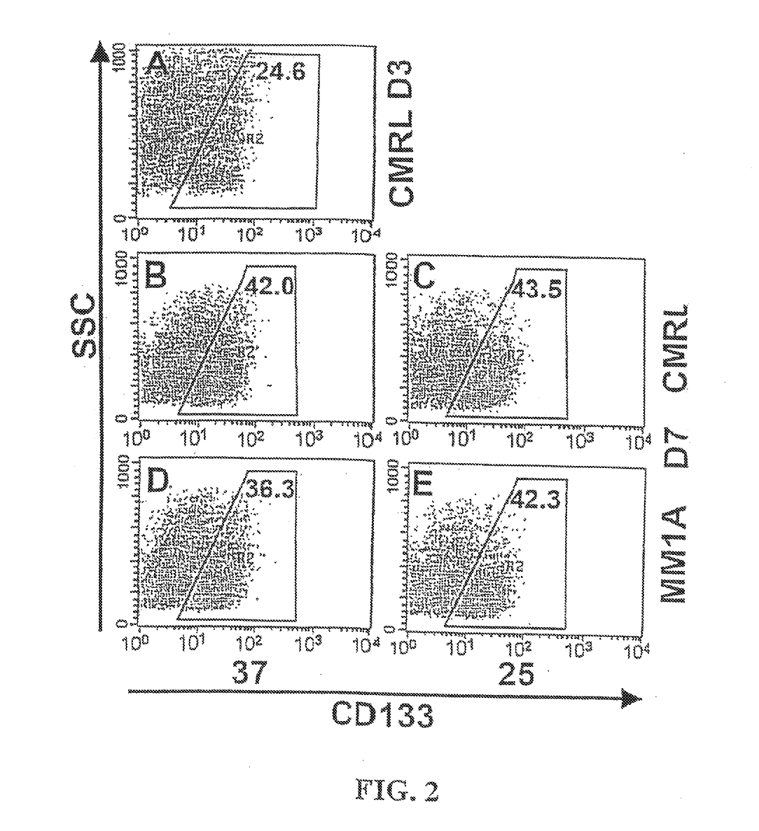

Islet preparation tissue was routinely cultured in serum-free Miami Media 1A (MM1A) at 37° C. Common alternative conditions use serum-supplemented CMRL 1066 (CMRL) and culture at 25° C. prior to transplantation. On D3 (65% islets) the culture was 24.6% CD133+. The fraction of cells expressing CD133 increased between 1.5- and 1.8-fold under all conditions (FIG. 2).

### Example 5

### CD133+ Population Increases in the Context of ADM and is Enhanced by Inhibition of Notch Activation

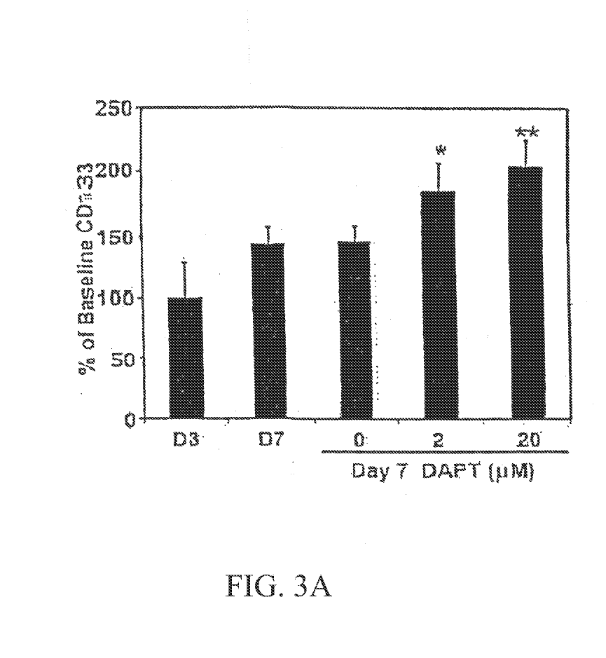

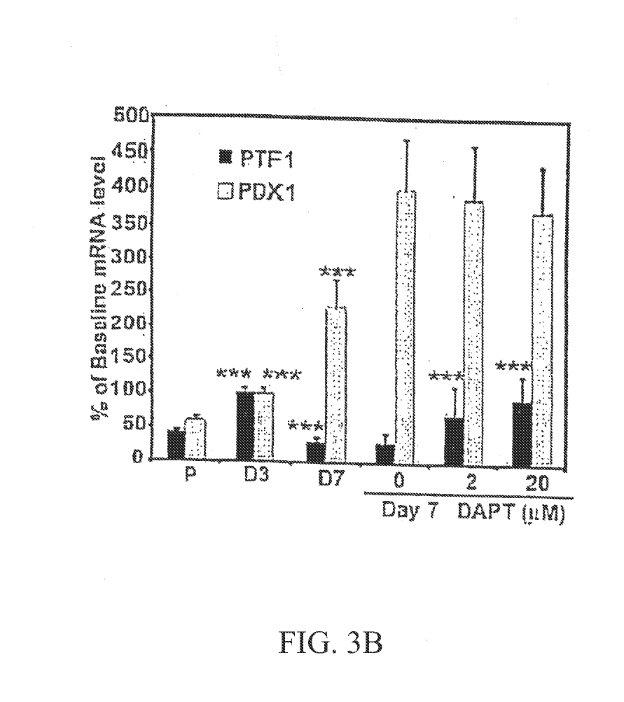

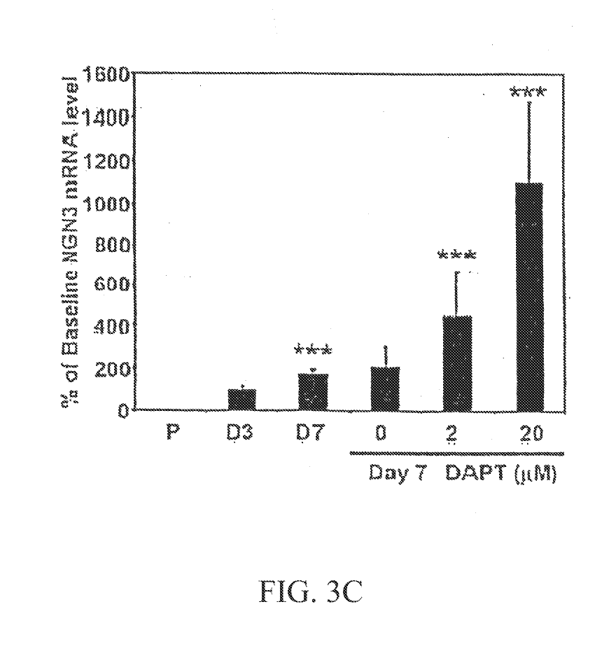

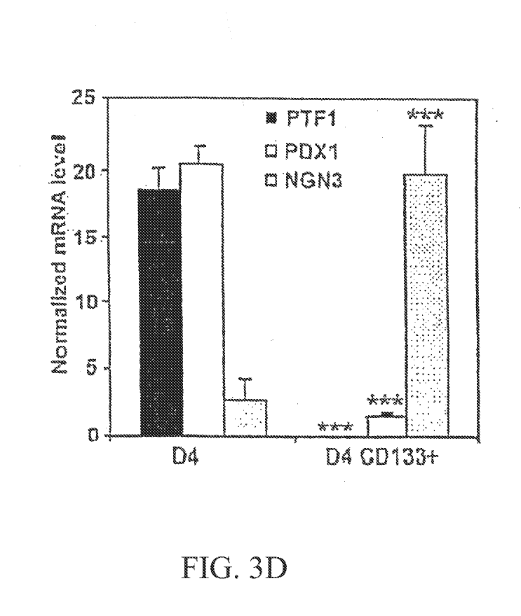

The expression of CD133, CK19, the acinar cell marker amylase (AMY) and Notch regulated genes was investigated in two independent islet preparations (70% and 85% islets) cultured between D3 and 7 in MM1A and CMRL media. On D3, 24.6% and 16.4% of cells were CD133+ in these two preparations. When data from both preparations in both media types were combined, the % CD133+ cells on D7 increased to a mean of 144±14.1% of D3 values (FIG. 3A). On D3, 35.5±7.3% and 36.1±9.6% of cells expressed AMY and CK19, respectively. By D7, the % AMY+ cells decreased significantly (9.1-fold, P<0.001) to 3.9±6.9% while the % CK19 expressing cells increased significantly (1.7-fold, P<0.001) to 61.6±9.3% (FIG. 4).

To determine if Notch signaling played a role in the accumulation of CD133+ cells in these cultures we treated tissue with DMSO carrier alone, 2 micromolar or 20 micromolar gamma-secretase inhibitor DAPT (Dovey H F et al., Functional gamma-secretase inhibitors reduce beta-amyloid peptide levels in brain. J Neurochem 76:173-181, 2001). The mean % CD133+ cells was significantly higher in 2 micromolar (1.3-fold, P<0.05) and 20 micromolar DAPT (1.4-fold, P<0.01) than in carrier alone (FIG. 3A). There was a statistically significant increase in AMY expression (6.0-fold, P<0.05) from 2.9±3.8% in carrier alone to 17.5±17.1% in 20 micromolar DAPT. There was no significant difference in the percentage of cells expressing CK19 in DAPT (FIG. 4).

To confirm that Notch signaling was active and specifically inhibited by DAPT, the mRNA expression of the Notch target gene Hairy and Enhancer of Split 1 (HES1) was investigated. HES1 mRNA levels decreased significantly compared to carrier alone, by 1.9-fold (P<0.001) and 2.0-fold (P<0.01) in 2 micromolar and 20 micromolar DAPT, respectively. There were no significant differences in HES1 mRNA expression between media types. To establish that the increased percentage of CD133+ cells was due to inhibition of Notch activation rather than other non-specific effects of gamma-secretase inhibition, an independent replica experiment was conducted on a preparation of 75% islet purity cultured in MM1A. In addition to DAPT, JKL6, a gamma-secretase inhibitor that does not inhibit the Notch pathway (Petit A et al., JLK isocoumarin inhibitors: selective gamma-secretase inhibitors that do not interfere with notch pathway in vitro or in vivo. J Neurosci Res 74:370-377, 2003) was used. In MM1A alone, the percentage of CD133+ cells increased from 23.1% to 39.4% between D3 and 7 (FIG. 5). The % CD133+ cells increased with increasing DAPT concentration from 40.8% in carrier to a maximum of 54.8% in 10 micromolar DAPT. No increase in the percentage of CD133+ was seen in cultures treated with JKL6 (FIG. 5), however, there was a small increase in normalized HES1 mRNA level at 0.1 micromolar JKL6 (1.1-fold) and a small decrease (1.1-fold) at 10 micromolar JKL6, compared to carrier only control.

An increase in CD133+ cells over time and in response to DAPT has been observed in more than 15 independent cultures. However, in two instances, cultures failed to increase in the percentage of CD133+ cells or respond to DAPT.

### Example 6

### Notch Signaling Represses PTF1 and NGN3 mRNA Levels

Pancreas specific transcription factor, 1a (PTF1) plays critical roles in formation and spatial organization of the murine exocrine and endocrine pancreas (Krapp A et al., The bHLH protein PTF1-p48 is essential for the formation of the exocrine and the correct spatial organization of the endocrine pancreas. Genes Dev 12:3752-3763, 1998) and marks precursor cells that give rise to all exocrine and most endocrine cells (Kawaguchi Y et al., The role of the transcriptional regulator Ptf1a in converting intestinal to pancreatic progenitors. Nat Genet 32:128-134, 2002). In mouse (Fukuda A et al., Ectopic pancreas formation in Hes1-knockout mice reveals plasticity of endodermal progenitors of the gut, bile duct, and pancreas. J Clin Invest 116:1484-1493, 2006) and zebrafish (Esni F et al., Notch inhibits Ptf1 function and acinar cell differentiation in developing mouse and zebrafish pancreas. Development 131:4213-4224, 2004) PTF1 is negatively regulated by HES1. The impact of culture and inhibition of Notch signaling on the mRNA expression levels of human PTF1 was investigated in the two independent islet preparations cultured in both MM1A and CMRL media. The mRNA expression level of PTF1 was significantly higher (2.3-fold, P<0.001) in islet preparation material on D3 than in the intact adult pancreas. By D7, PTF1 mRNA levels decrease significantly (3.3-fold, P<0.001) as compared to levels on D3. PTF1 mRNA levels increased significantly in 2 micromolar (2.3-fold, P<0.001) and 20 micromolar (3.0-fold, P<0.001) DAPT as compared to carrier (FIG. 3B).

Like PTF1, pancreatic and duodenal homeobox 1 (PDX1) plays an early and necessary role in pancreatic development and is required for maintenance of function, but is not known to be directly regulated by Notch. In the two independent islet preparations, PDX1 mRNA expression increased significantly between D3 and 7 (2.3-fold, P<0.001) but was not significantly affected by DAPT (FIG. 3B).

Neurogenin 3 (NGN3) defines a subset of progenitors that give rise to endocrine cells and is both necessary and sufficient to drive islet formation during development (Lee J C et al., Regulation of the pancreatic pro-endocrine gene neurogenin3. Diabetes 50:928-936, 2001). NGN3 is negatively regulated by HES1. NGN3 mRNA was detected at very low levels (0.05% of D3) in the adult pancreas. On D3, NGN3 mRNA levels were ˜2.7-fold lower than human fetal pancreas positive control RNA. There was a significant increase (1.7-fold, P<0.001) in NGN3 mRNA level between D3 and 7 of our cultures. NGN3 mRNA expression levels increased in the presence of 2 micromolar (2.1-fold, P<0.001) and 20 micromolar (5.2-fold, P<0.001) DAPT as compared to carrier alone (FIG. 3C). There were significant differences in NGN3 mRNA levels between media. In carrier alone, the level of NGN3 mRNA was higher (2.1-fold, P<0.001) in CMRL media than MM1A. However, in the presence of 2 micromolar (1.7-fold, P<0.05) and 20 micromolar DAPT (1.9-fold, P<0.001), NGN3 mRNA levels were higher in MM1A than CMRL.

### Example 7

CD133+ Cells Express NGN3

Following immunomagnetic bead enrichment for CD133 on a D4 islet preparation (40% islet purity) NGN3 mRNA levels were significantly higher (7.5-fold, P<0.001) in the CD133-enriched population (>98% CD133+) compared to the unenriched population (˜25% CD133+) and were 11.6-fold greater than in human fetal pancreas. The CD133-enriched population expressed significantly less PTF1 mRNA (>1500-fold, P<0.001) and PDX1 mRNA (12.7-fold, P<0.001) than unenriched (FIG. 3D). Immunohistochemical staining of the D4 tissue for PDX1 indicated very low to undetectable levels of PDX1 in CD133+ cells. This is also true of D7 tissues. No definitive CA19.9/NGN3 coexpression was observed in D7 tissue cultured in either MM1A or CMRL media, however, CA19.9/NGN3 coexpression was observed in the presence of 20 micromolar DAPT in both media conditions.

To determine the effects of culture and gamma-secretase inhibition on the expression of NGN3 protein and the extent to which CD133+ cells express NGN3 protein, frozen sections of tissue from the two independent islet isolations were stained with antibodies to CD133/2 and NGN3. NGN3 protein expression was not detected in intact adult pancreas controls or on D3 of culture. On D7, NGN3 protein was detected within the nucleus of 23.4±6.3% and 37.9±8.5% of cells in MM1A and CMRL media, respectively. The percentage of cells expressing NGN3 in the presence of 20 micromolar DAPT were 37.8±8.3 and 43.0±9.9 in MM1A and CMRL media, respectively. In a third independent islet isolation (45% islets) cultured from D3 to 7 in MM1A, 15.4±8.7% and 26.3±8.3% of nuclei were NGN3+ on D7 in the absence and presence of 20 micromolar DAPT, respectively. Although the mean % NGN3+ cells was consistently higher in the presence of DAPT, this difference was only statistically significant (P<0.01) in MM1A media from the first experimental series. Cells coexpressing NGN3 and CD133 were identified at D7 in the absence and presence of 20 micromolar DAPT. NGN3+/CD133− and NGN3−/CD133+ cells were detected under all treatment conditions. Isotype negative controls showed no staining above background.

To evaluate the ability of isolated CD133+ cells to proliferate in vitro, a D3 islet preparation (45% islets) was disaggregated, immunomagnetically enriched for CD133 expression and plated at low cell density in a media formulation used to support in vitro beta cell neogenesis from rat exocrine tissue (Baeyens Let al., In vitro generation of insulin-producing beta cells from adult exocrine pancreatic cells. Diabetologia 48:49-57, 2005). Under these conditions, 38.8±5.9% of cells adhered after 12 hrs. Over the next 8 days, single cells proliferated to form colonies with a mean cell number of 13.3±7.3 (3.7 population doublings) then stopped dividing. At this point, virtually all cells express NGN3. Insulin and insulin C-peptide expression were not detected.

Moreover, CD133+ cells from such cultures show the ability to form spheres in vitro, a characteristic of other CD133 expressing progenitor cell populations. These spheres form most readily in modified HuES media [KO-DMEM, 1× pen/strep, 1× glutamax, 1× NEAA, 10% KO serum replacement, 0.1×2-mercaptoethanol, 1× N2 supplement (Invitrogen), 10% Plasmanate (Bayer), Fibroblast growth factor 2 (20 ng/ml), Leukemia inhibitory factor, (10 ng/ml), epidermal growth factor (20 ng/ml)] or in PS media [DMEM/F12, 1× pen/strep, 1× glutamax, 1× N2 supplement (Invitrogen), Fibroblast growth factor 2 (20 ng/ml), Leukemia inhibitory factor, (10 ng/ml), epidermal growth factor (20 ng/ml)] that has been conditioned by overnight incubation with human embryoid body derived cell line SDEC (Shamblott et al., Proc Natl Acad Sci USA. 2001; 98:113-8). An additional growth factor that has shown early promise in the expansion of these cells is platelet derived growth factor-BB (PDGF-BB) at 20 ng/ml. Thus, further demonstration of the endocrine progenitor cell nature of the CD133+ cells identified and utilized herein is provided by the expression of NGN3 and the response to DAPT.

### Example 8

### Implantation of CD133+ Cells

Pancreas tissue is obtained from a patient undergoing surgery for acute pancreatitis is utilized in preparing pancreatic endocrine progenitor cells for reimplantation. Using magnetic beads to which anti-CD133 antibody is bound, CD133+ cells are separated from a population of pancreatic cells which are disaggregated from the human pancreas tissue, as is described in Examples 1 and 2. The cells are expanded in culture using Miami Medium 1A. After 7 days in culture, the expanded progenitor cell population is administered into the patient's vasculature by intravenous infusion, to provide replacement of pancreatic endocrine cell function.

While certain features of the invention have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

What is claimed is:

1. A method for identifying pancreatic endocrine progenitor cells in a cellular sample or tissue sample comprising exposing the cellular sample or tissue sample to at least one detectable agent that binds to cells expressing a glycosylated form of a prominin-1 gene product, thereby identifying pancreatic endocrine progenitor cells therein.

2. The method of claim 1 wherein the at least one detectable agent is a detectably labeled primary binding partner that binds to a glycosylated form of a prominin-1 gene product, or a combination of an unlabeled primary binding partner that binds to a glycosylated form of a prominin-1 gene product and a detectably labeled secondary binding partner that binds to the unlabeled primary binding partner.

3. The method of claim 1 wherein the at least one detectable agent comprises at least one antibody or lectin that binds to a glycosylated form of a prominin-1 gene product, binds to CD133, binds to epitope CD133/1, or binds to epitope CD133/2.

4. The method of claim 1 wherein the cellular sample is a pancreatic islet preparation.

5. The method of claim 1 wherein the cellular sample is human pancreatic tissue.

6. A method for isolating pancreatic endocrine progenitor cells from a cellular population comprising identifying cells expressing a glycosylated form of a prominin-1 gene product therein and separating said cells from the cellular population, thereby isolating pancreatic endocrine progenitor cells.

7. The method of claim 6 wherein the identifying comprises exposing the cellular population to at least one detectable agent that labels cells that express a glycosylated form of a prominin-1 gene product.

8. The method of claim 7 wherein the at least one detectable agent is a detectably labeled primary binding partner that binds to a glycosylated form of a prominin-1 gene product, or a combination of an unlabeled primary binding partner that binds to a glycosylated form of a prominin-1 gene product and a detectably labeled secondary binding partner that binds to the unlabeled primary binding partner.

9. The method of claim 8 wherein the at least one detectable agent comprises at least one antibody or lectin that binds to a glycosylated form of a prominin-1 gene product, binds to CD133, binds to epitope CD133/1, or binds to epitope CD133/2.

10. The method of claim 6 wherein the separating is performed by fluorescence activated cell sorting.

11. The method of claim 6 wherein the separating is by using magnetic beads that bind to cells expressing a glycosylated form of a prominin 1 gene product, isolating the magnetic beads to which said cells are bound, then releasing said cells bound thereto, thereby isolating pancreatic endocrine progenitor cells.

12. The method of claim 6 wherein the cellular population is obtained from a pancreatic islet preparation.

13. The method of claim 6 wherein the cellular population is obtained from human pancreatic tissue.

14. A method for treating a patient in need of pancreatic endocrine replacement therapy comprising isolating pancreatic endocrine progenitor cells from a cellular population in accordance with claim 6, then administering the isolated pancreatic endocrine progenitor cells to the patient.

15. The method of claim 14 wherein the patient has insulin-dependent diabetes mellitus or is post-pancreatic surgery insulin deficient.

16. The method of claim 14 wherein the cellular population is obtained from a pancreas HLA matched to the subject.

17. The method of claim 14 wherein the isolated pancreatic endocrine progenitor cells are cultured or expanded in vitro prior to administration.

18. The method of claim 15 wherein the pancreatic endocrine progenitor cells are isolated from pancreatic tissue obtained from the same post-pancreatic surgery patient.

19. A system for isolating or enriching pancreatic endocrine progenitor cells from a cellular sample comprising:
a. means for detecting cells expressing a glycosylated form of a prominin-1 gene product therein, and
b. means for separating the cells expressing a glycosylated form of a prominin-1 gene product therein from other cells in the cellular sample.

20. A method for treating a patient in need of pancreatic endocrine replacement therapy comprising isolating or enriching pancreatic endocrine progenitor cells from a cellular population in accordance with the system of claim 19, then administering the isolated pancreatic endocrine progenitor cells to the patient.

21. Pancreatic endocrine progenitor cells prepared by the process of obtaining a cellular population comprising pancreatic cells, identifying cells expressing a glycosylated form of a prominin 1 gene product therein, then separating said cells from the cellular population; thereby providing isolated pancreatic endocrine progenitor cells.
